(19) United States
(12) Patent Application Publication
(10) Pub. No.: US 2002/0016979 A1
(43) Pub. Date: Feb. 7, 2002

(54) MAIZE PLANTS EXHIBITING MYCORRHIZAL RESPONSIVENESS AND AN ENHANCED ABILITY TO EXTRACT PHOSPHORUS FROM SOIL

(76) Inventors: SHAWN M. KAEPPLER, OREGON, WI (US); JENNIFER L. PARKE, CORVALLIS, OR (US)

Correspondence Address: ROCKEY MILNAMOW & KATZ, TWO PRUDENTIAL PLAZA SUITE 4700, 180 NORTH STETSON AVENUE, CHICAGO, IL, 60601

(21) Appl. No.: 09/257,399

(22) Filed: Feb. 25, 1999

Related U.S. Application Data
(60) Provisional application No. 60/076,185, filed on Feb. 27, 1998.

(51) Int. Cl. C12N 15/82

(52) U.S. Cl. 800/278

(57) ABSTRACT

The present invention relates to maize plants having within their genomes, quantitative trait loci which confer the traits of mycorrhizal responsiveness and/or an ability to extract phosphorus from soil and translocate the extracted phosphorus from the soil and into the plant.

### RELATED APPLICATION INFORMATION

The present application is a continuation-in-part of U.S. application Ser. No. 60/076,185, filed on Feb. 27, 1998.

### FEDERAL FUNDING INFORMATION

This invention was made with United States government support awarded by the following agencies: USDA Hatch 3919; 5211; 7201. The United States has certain rights in this invention.

### TECHNICAL FIELD OF THE INVENTION

The present invention relates to maize plants having within their genome, quantitative trait loci which confer the traits of mycorrhizal responsiveness and/or an ability to extract phosphorus from soil and translocate the extracted phosphorus from the soil and into the plant.

### BACKGROUND OF THE INVENTION

Maize, also known as corn, has been used as a source of food for human and animal consumption. Today, maize supplies about twenty percent of the world's calories. Fertilization of maize production fields with inorganic phosphorus (P), nitrogen (N), and potassium (K) has been one of the major factors in the increased and stabilized yields realized in the United States over the last sixty years.

The application of fertilizer is one of the major on-farm production costs associated with maize. Nitrogen application costs approximately $25.00 per acre assuming an application of 130 pounds per acre, and a cost of $310.00 per ton of 82% anhydrous nitrogen. Phosphorus application costs approximately $15.00 per acre assuming an application of 56 pounds per acre, and a cost of $294.00 per ton of 46% superphosphate. Potassium applications cost approximately $10 per acre (Application rates and fertilizer cost estimates are from the 1996-97 United States Department of Agriculture-Agriculture Resources and Economic Indicators (USDA-AREI)). Based on a yield of 135 bushels per acre and a grain price of from about $2.50 to about $3.00 per bushel, fertilization with nitrogen, phosphorus and potassium accounts for about 12 to about 15 percent of gross income.

Additionally, the use of fertilizer has further economic and environmental costs. With respect to the economic costs, the supply of high-grade phosphate reserves in the U.S. are forecast to decline as quality reserves in Florida, North Carolina and Idaho become depleted (see the 1996 United States Geological Survey Statistical Compendium). This will lead to increased imports of phosphate from North Africa, which will negatively affect the U.S. trade balance and the stability of supply.

With respect to the environmental costs, in 1993, the production and application of fertilizers and pesticides accounted for over 40% of all energy used in agriculture (see the 1996-1997 USDA-AREI). Much of this energy is derived from non-renewable fossil fuels. In addition, phosphorus and nitrogen run-off causes eutrophication in lakes and streams, a cycle of algal blooms leading to reduced water quality, depletion of oxygen by decomposing bacteria, followed by die-off of fish and other organisms. The Environmental Protection Agency has estimated that nutrient pollution is the leading cause of water quality impairment in lakes and estuaries and the third leading cause in streams. The economic impact of non-point run-off is billions of dollars per year, in addition to the loss in aesthetic value.

Phosphorus is essential for plant growth and is required in large amounts by plants. Plants need phosphorus throughout their life cycle, but especially during early growth stages for cell division. Phosphorus is mobile in plants. Specifically, phosphorus is absorbed during early growth and is later redirected for use in seed formation. Phosphorus is present in soil and in fresh water only in micromolar concentrations in large part because phosphorus is commonly bound to many soil constituents that make it unavailable or only sparingly available to plants (Lynch et al., HortScience, 30(6) 1165-1171 (1995)). An additional problem is that the phosphate cycle in most terrestrial ecosystems is open-ended and tends toward depletion, unlike the nitrogen cycle, in which the atmosphere provides continual inputs to the soil. Id. Thereupon, in many soil types, the application of phosphorus will always be necessary.

Mutualistic associations between plant roots and fungi, known as mycorrhizas, are extremely widespread (Hoffman, C. A., et al., Annu. Rev. Ecol. Sys. 26:69-92 (1995)). In agriculture, the most important mycorrhizas are the zygomycetous fungi (Order Glomales), generally referred to as vesicular-arbuscular mycorrhizae (VAM) or arbuscular mycorrhizae (AM). Arbuscular mycorrhizal fungi are present and mediate plant root and soil interactions in most terrestrial ecosystems and crop production systems (George, E., et al., Crit. Rev. Biotech. 15(3/4):257-270 (1995)). Arbuscular mycorrhizal fungi have been shown in natural grassland ecosystems to greatly enhance the uptake of phosphorus, as well as other nutrients (Hooker, J. E., et al., Crit. Rev. in Biotech. 15:201-212 (1995)). It is believed that the enhanced uptake is due to the large increase in absorptive surface area and in increased soil volume explored by the network of mycorrhizal mycelium.

Arbuscular mycorrhizal fungi associate with most major crop species with the exception of Brassiceae, Chenopodiaceae, and Polygonaceae (Hoffman, C. E., et al., Annu. Rev. Ecol. Syst., 26:69-92 (1995)). Generally, the association of AM fungi with crop plants is not of major importance in current production systems due to high levels of chemical fertilizer inputs and the tendency of tillage to disrupt hyphal networks (Hooker, J. E., et al., Crit. Rev. in Biotech. 15:201-212 (1995)). However, there is an increasing movement toward minimum tillage systems with reduced fertilizer inputs. Reduced input and tillage systems could potentially greatly benefit from AM symbiosis.

There is a need in the art for new varieties of maize that have an enhanced ability to extract phosphorus in the soil and translocate the extracted phosphorus from the soil and into the plant. Such plants would require less applied phosphorus, thus benefitting growers by reducing the costs associated with applying fertilizer. Furthermore, because growers would be applying less phosphorus to their maize crops, less phosphorus would run-off into lakes and streams, thereby benefitting the environment.

### SUMMARY OF THE INVENTION

In one embodiment, the present invention relates to an improved maize plant having a genome that contains quantitative trait loci which confer to the plant an ability to extract phosphorus from soil and translocate the extracted phosphorus into the plant. The quantitative trait loci in the maize plant that confer this trait are located on chromosome 1, interval 186 to 212 as shown in FIG. 1, chromosome 7, interval 46 to 74 as shown in FIG. 1, chromosome 8, interval 88 to 106 as shown in FIG. 1 and chromosome 9, interval 56 to 62 as shown in FIG. 1.

In another embodiment, the present invention relates to an improved maize plant having a genome that contains quantitative trait loci which confer to the plant mycorrhizal responsiveness when the plant is grown in soil containing mycorrhiza. The quantitative trait loci in the maize plant that confer this trait are located on chromosome 2, interval 160 to 178 as shown in FIG. 1. chromosome 5, interval 0 (the interval end) to 8 as shown in FIG. 1, chromosome 7, interval 46 to 74 as shown in FIG. 1 and chromosome 9, interval 50 to 72 as shown in FIG. 1.

An improved maize plant having a genome which confers to the plant an ability to extract phosphorus from soil and translocate the extracted phosphorus into the plant can contain within its genome, quantitative trait loci from a first donor maize line parent, as well as quantitative trait loci associated with the ability to extract phosphorus from the soil and translocate the extracted phosphorus into the plant from a second donor maize line parent. The second donor maize line parent contains quantitative trait loci located in on chromosome 1, interval 186 to 212, chromosome 7, interval 46 to 74, chromosome 8, interval 88 to 106, and chromosome 9, interval 56 to 62. Preferably, the first donor maize line parent is other than Mo17 and the second donor maize line parent is Mo17 or progeny thereof.

An improved maize plant having a genome which confers to the plant the trait of mycorrhizal responsiveness when the plant is grown in soil containing mycorrhiza, contains within its genome, quantitative trait loci from a first donor maize line parent, as well as quantitative trait loci associated with mycorrhizal responsiveness from a second donor maize line parent. The second donor maize line parent contains quantitative trait loci located on chromosome 2, interval 160 to 178, chromosome 5, interval 0 to 8, chromosome 7, interval 46 to 74, and chromosome 9, interval 50 to 72. Preferably, the first donor maize line parent is other than B73 and the second donor maize line parent is B73 or progeny thereof.

A maize plant having a genome which confers to the plant the traits of mycorrhizal responsiveness and an ability to extract phosphorus from the soil and translocate the extracted phosphorus into the plant when the plant is grown in soil containing mycorrhiza contains within its genome, quantitative trait loci associated with mycorrhizal responsiveness from a first donor maize line parent B73, or progeny thereof, as well as quantitative trait loci associated with an ability to extract phosphorus from soil and translocate the extracted phosphorus into the plant, from a second donor maize line parent, Mo17, or progeny thereof. The quantitative trait loci conferring the traits of mycorrhizal responsiveness and extraction of phosphorus from the soil and translocation of this extracted phosphorus into the plant, are located on chromosome 2, interval 160 to 178, chromosome 5, interval 0 to 8, chromosome 7, interval 46 to 74, and chromosome 9, interval 50 to 72 for mycorrhizal responsiveness and on chromosome 1, in interval 186 to 212, chromosome 7, interval 46 to 74, chromosome 8, interval 88 to 106, and chromosome 9, interval 56 to 62 for extraction of phosphorus from the soil and translocation of the extracted phosphorus into the plant.

The present invention also relates to methods for producing an improved inbred maize plant, which when crossed with a second inbred maize plant, produces a hybrid maize plant which exhibits the traits of mycorrhizal responsiveness and/or an ability to extract phosphorus from the soil and translocate the extracted phosphorus into the plant.

In one embodiment, the method involves selecting a first donor maize parental line having a genome which exhibits mycorrhizal responsiveness when the plant is grown in soil containing mycorrhiza and/or an ability to extract phosphorus from the soil and translocate the extracted phosphorus into the plant. The first donor maize parental line contains quantitative trait loci for either or both of these traits which are located on chromosome 2, interval 160 to 178, chromosome 5, interval 0 to 8, chromosome 7, interval 46 to 74, and chromosome 9, interval 50 to 72 for mycorrhizal responsiveness and on chromosome 1, in interval 186 to 212, chromosome 7, interval 46 to 74, chromosome 8, interval 88 to 106, and chromosome 9, interval 56 to 62 for extraction of phosphorus from the soil and translocation of the extracted phosphorus into the plant. A second donor maize parental line having desirable phenotypic traits is then selected. The first donor maize parental line is crossed with the second donor maize parental line to produce a segregating plant population. The plant population is screened for one or more quantitative trait loci associated with the traits of mycorrhizal responsiveness and/or the ability to extract phosphorus from the soil and translocation of the extracted phosphorus into the plant. Plants are then selected from the population having the identified quantitative trait loci for further crossings until a maize line is obtained which is homozygous for the traits of mycorrhizal responsiveness and/or the ability to extract phosphorus from the soil and translocate the extracted phosphorus into the plant. For mycorrhizal responsiveness, the first donor maize parental line can be B73. For the ability to extract phosphorus from the soil and translocate the extracted phosphorus to the plant, the first donor maize parental line can be Mo17.

In another embodiment, the present invention relates to methods of producing hybrid maize plants having a genome containing quantitative trait loci which confer to the plant the traits of mycorrhizal responsiveness and/or an ability to extract phosphorus from the soil and translocate the extracted phosphorus into the plant. The method involves crossing an inbred maize line that is homozygous for the traits of mycorrhizal responsiveness and/or an ability to extract phosphorus from soil and translocate the extracted phosphorus into the plant with a donor maize parental line to produce a segregating population. The donor maize parental line is an inbred line that contains desirable phenotypic traits or is an inbred maize line that is homozygous for the traits of mycorrhizal responsiveness and/or an ability to extract phosphorus from soil and translocate the extracted phosphorus into the plant. The segregating plant population is then screened for one or more quantitative trait loci associated with the traits of mycorrhizal responsiveness and/or an ability to extract phosphorus from the soil and translocate the extracted phosphorus into the plant. Hybrid plants exhibiting the traits of mycorrhizal responsiveness and/or the ability to extract phosphorus from the soil and translocate the extracted phosphorus from the soil and into the plant are then selected.

### BRIEF DESCRIPTION OF THE FIGURES

FIG. 1A through 1B show a map of chromosomes 1 to 10 produced from a B73×Mo17 recombinant population. For each chromosome, the names of a marker are positioned to the right of the vertical line. The numbers to the left of the vertical lines are the relative marker positions in the centiMorgan (cM). CentiMorgans are an unit for measuring genetic distance.

FIG. 2 shows a listing of the molecular markers used to identify the quantitative trait loci of the present invention. The molecular markers used to identify the quantitative trait loci of the present invention are: umc156a, umc127c, umc90, umc65a, asg49, umc245, Bln2.369 and csu31. The remaining molecular markers shown in FIG. 2 define chromosome regions that are not associated with mycorrhizal responsiveness and the ability to extract phosphorus from soil and translocate the extracted phosphorus into a plant.

### DETAILED DESCRIPTION OF THE INVENTION

Phosphorus is an essential element required in large amounts by plants throughout their life cycle. Plants obtain phosphorus from the soil. The amount of free phosphorus available for plants in the soil is limited to micromolar concentrations. A large proportion of phosphorus in soils is not biologically available. Approximately one-half of the unavailable phosphorus is in organic forms such as decomposing plant tissue. The other portion is in mineral complexes with calcium or other soil ions.

In one embodiment, the present invention relates to improved maize plants exhibiting the traits of mycorrhizal responsiveness and/or an ability to extract phosphorus from the soil and translocate the extracted phosphorus from the soil and into the plant. More specifically, the present invention relates to the introgression into a maize plant of genetic material which confers upon the maize plant the traits of mycorrhizal responsiveness and/or the ability to extract phosphorus contained in soil and translocate the extracted phosphorus from the soil and into the plant. The phosphorus contained in the soil to be extracted may be in free form or bound to soil constituents such as mineral complexes. The maize plants of the present invention can be inbreds, hybrids or progeny thereof.

In another embodiment, the present invention relates to a method for producing an improved inbred maize plant which when crossed with a second inbred maize plant produces a hybrid maize plant that exhibits the traits of mycorrhizal responsiveness and/or the ability to extract phosphorus from the soil and translocate the extracted phosphorus from the soil and into the plant.

The improved maize plants of the present invention exhibit an environmentally friendly genotype. The improved maize plants of the present invention either extract phosphorus that is bound to soil constituents or possess the ability to increase the solubility of the free phosphorus present in the soil for enhanced uptake of the phosphorus by the roots of the plant. Unlike conventional plants, the plants of the present invention require less applied phosphorus in the form of fertilizer in order to grow and proliferate. Applying less fertilizer provides a number of benefits. First, the application of less phosphorus to plants benefits growers by reducing the amount of phosphorus that the growers will need to purchase in order to fertilize their plants. This provides a cost savings to the growers. A second benefit is that it reduces the amount of phosphorus depleted from existing United States phosphate reserves. A third benefit is that it reduces the damage caused to the environment by phosphate run-off.

The maize plants of the present invention were developed by locating and identifying specific quantitative trait loci which confer upon a maize plant the traits of mycorrhizal responsiveness and the ability to extract phosphorus from the soil and translocate the extracted phosphorus from the soil and into the plant. As used herein, the term “mycorrhizal responsiveness” means the relative growth of a genotype in the presence of mycorrhizal infection compared to the growth of that genotype in the absence of mycorrhizal infection. Mycorrhizal responsiveness is expressed as a percent. A plant exhibiting mycorrhizal responsiveness will have an increased ability to uptake phosphorus from the soil for use in the plant. The plants of the present invention exhibit mycorrhizal responsiveness to arbuscular mycorrhiza. Arbuscular mycorrhiza are endomycorrhiza which form an intracellular association with a host plant. Examples of abuscular mycorrhiza include Glomus etunicatum, G. claroideum, G. clarum, and Acaulospora mellea. As used herein, the term “quantitative trait loci/locus” or “QTL” means an identified region on a chromosome that is defined by specific molecular markers which spans a defined map interval and has an effect on a specific trait. As used herein, the term “molecular marker loci/locus” means the location on a genetic map or physical chromosome that is defined by a molecular marker. As used herein, the term “molecular marker(s)” means restriction fragment length polymorphism (RFLP), microsatellite, or an isozyme marker which defines a specific genetic and chromosome location. As used herein, the term “translocate” or “translocation” means the uptake and movement of nutrients or ions throughout a plant.

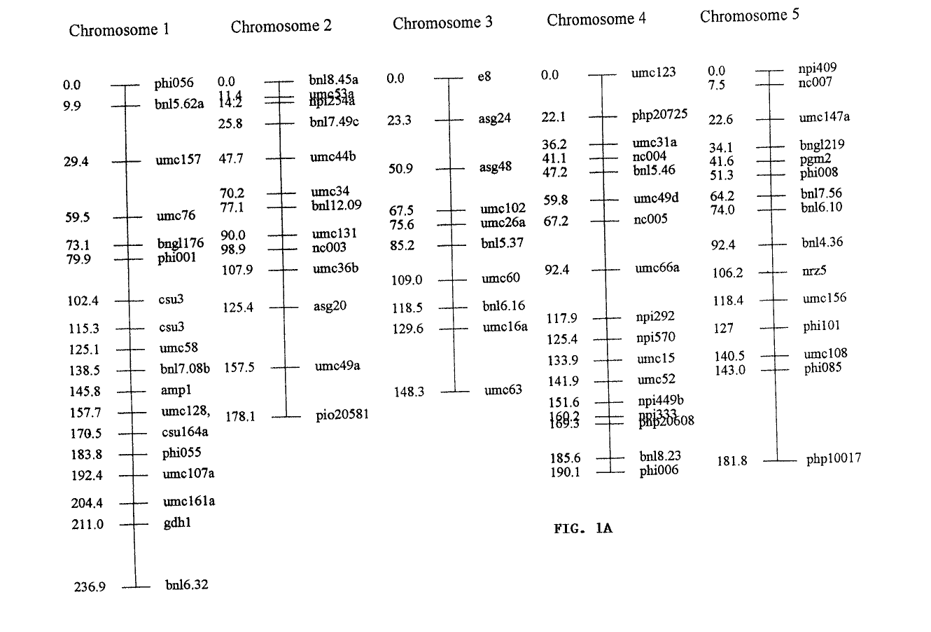

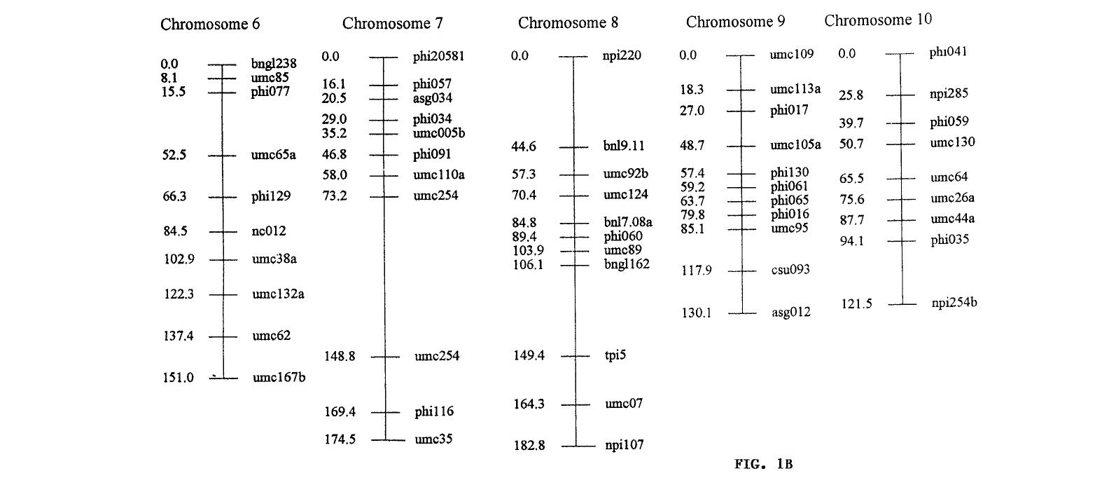

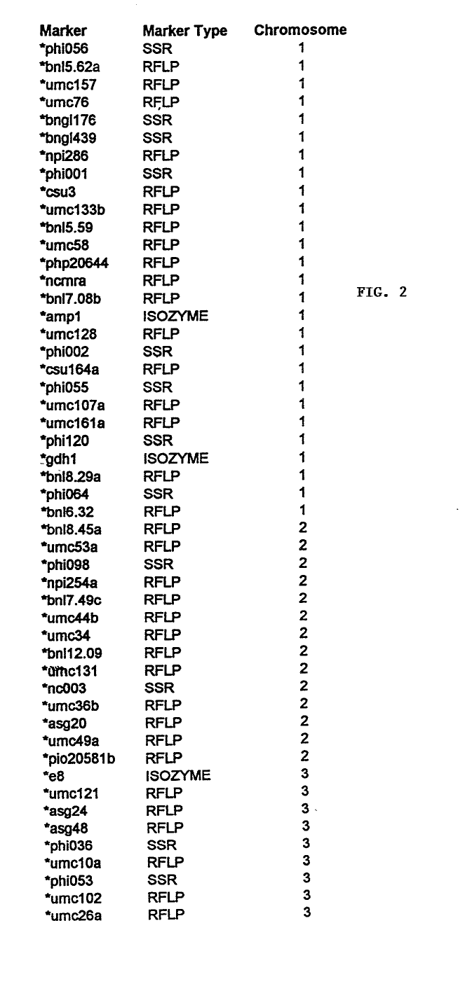

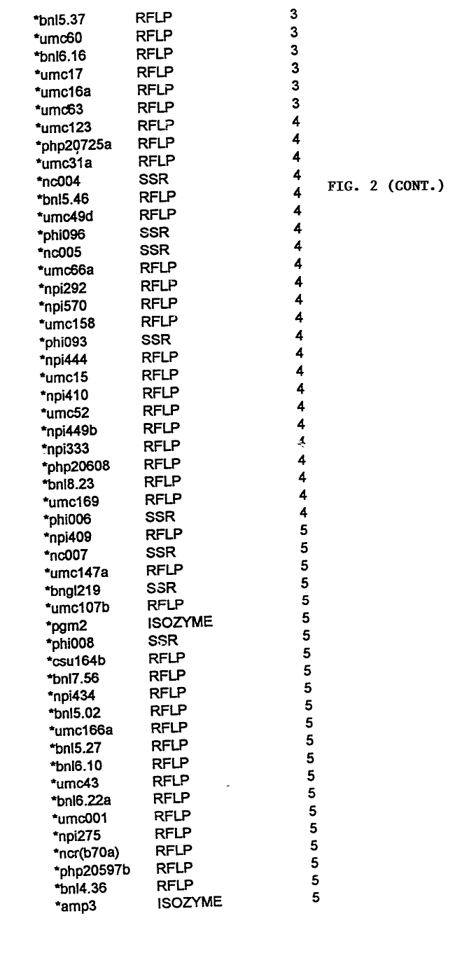

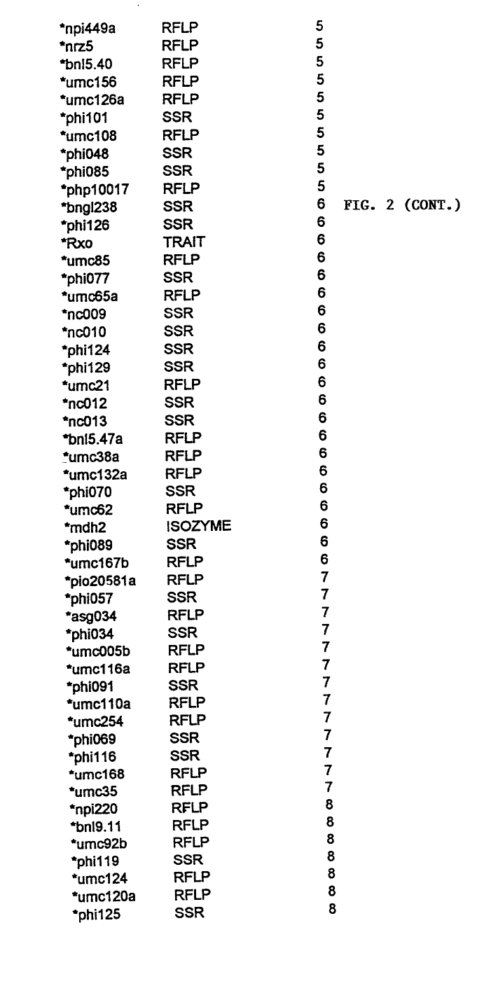

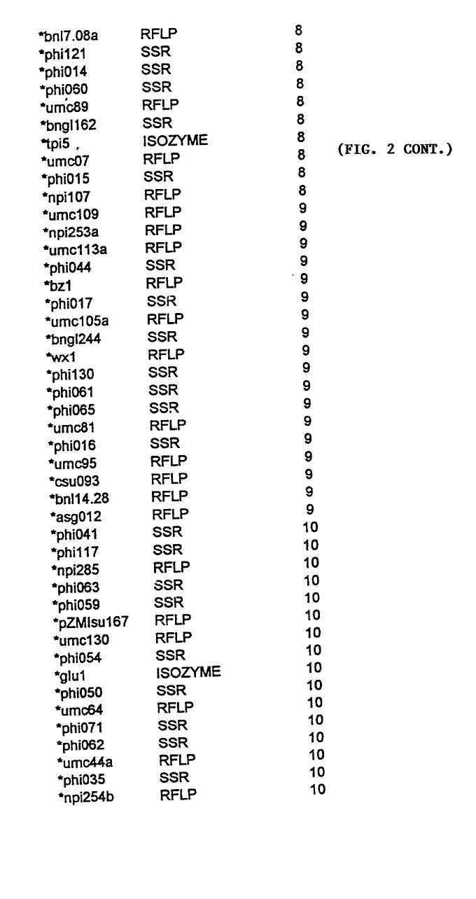

Four (4) quantitative trait loci have been discovered which confer the trait mycorrhizal responsiveness to a plant. These quantitative trait loci are referred to herein as RESPONSE1 to RESPONSE4 and are located on chromosome 2, interval 160 to 178, chromosome 5, 0 to 8, chromosome 7, interval 46 to 74, and chromosome 9, interval 50 to 72, respectively. As used herein, the term “interval” refers to a segment of the genetic map in centiMorgans as shown in FIG. 1. It has been discovered that each of these quantitative trait loci confer mycorrhizal responsiveness to a plant grown in soil containing mycorrhiza independently of each other. In addition, four (4) quantitative trait loci have been discovered which control the ability of maize plants to extract and translocate phosphorus from the soil. These quantitative trait loci are referred to herein LOW1 to LOW4 and are located on chromosome 1, interval 186 to 212, chromosome 7, interval 46 to 74, chromosome 8, interval 88 to 106, and chromosome 9, interval 56 to 62, respectively. Each of these quantitative trait loci is defined by the closest molecular marker and a confidence interval described in centiMorgans. These molecular markers are listed below in Table 1.

TABLE 1

| | Closest Molecular Marker and |
|---|---|
| Quantitative Trait Locus | Confidence Interval |
| RESPONSE1 | UMC49a, 160 to 178 |
| RESPONSE2 | NPI409, 0 (interval end) to 8 |
| RESPONSE3 | UMC110a, 46 to 74 |
| RESPONSE4 | PHI061, 50 to 72 |
| LOW1 | UMC107a, 186 to 212 |
| LOW2 | UMC110a, 46 to 74 |
| LOW3 | PHI060, 88 to 106 |
| LOW4 | PHI130, 56 to 62 |

The present invention contemplates improved inbred and hybrid maize plants, and progeny thereof, which exhibit mycorrhizal responsiveness when grown in soil containing mycorrhiza and have introgressed into their genome, genetic material from at least one, preferably more than one, and most preferably all of the quantitative trait loci hereinbefore described. The maize plants of the present invention are homozygous for one or more of the hereinbefore described quantitative trait loci. As used herein, the term “introgression” means the entry or introduction of a gene or quantitative trait loci from one plant into another plant. As used herein, the term “introgressing” means entering or introducing a gene or quantitative trait loci from one plant into another plant.

An inbred maize plant having a genome containing quantitative trait loci which confer the trait of mycorrhizal responsiveness to a plant when grown in soil containing mycorrhiza is B73. B73 contains all of the hereinbefore described quantitative trait loci. B73 is publically available from the National Seed Storage Lab (Fort Collins, Colo.). Other sources of this genetic material containing one or more of the hereinbefore described quantitative trait loci will of course be located since the present invention now allows this material to be identified. As shown in the examples, B73 grows poorly under low phosphorus conditions (17 mg kg⁻¹) in sterilized soil without mycorrhizae but exhibits about 6.5 times more growth when grown in the same soil with mycorrhizae.

The hereinbefore described quantitative trait loci have also been discovered to be responsible for conferring the ability to maize plants to extract phosphorus from soil and translocate the extracted phosphorus into a plant as shown below in the examples. It has been discovered that each of the hereinbefore described quantitative trait loci confers the ability to maize plants to extract phosphorus from the soil and translocate the extracted phosphorus into the plant independently of each other. The present invention also contemplates improved inbreds and hybrid maize plants, and progeny thereof, which exhibit the ability to extract phosphorus from soil and translocate the extracted phosphorus from the soil and into the plant. These plants have introgressed into their genome, genetic material from at least one, preferably more than one, and most preferably all of the hereinbefore described quantitative trait loci. Preferably, the maize plants of the present invention are homozygous for one or more of the hereinbefore described quantitative trait loci.

An inbred maize plant having a genome that contains genes which confer to a maize plant an ability to extract phosphorus from soil and translocate the extracted phosphorus from the soil and contains all of the hereinbefore described quantitative trait loci is Mo17. Mo17 is publically available from the National Seed Storage Lab (Fort Collins, Colo.). Other sources of this genetic material containing one or more of the hereinbefore described quantitative trait loci will of course be located since the present invention now allows this material to be identified. As shown in the examples, Mo17 exhibits an enhanced ability to extract phosphorus from soil and translocate the extracted phosphorus from the soil into the plant. Additionally, Mo17 exhibits very little mycorrhizal responsiveness.

The present invention also contemplates improved inbred and hybrid maize plants, and progeny thereof, which exhibit the traits of mycorrhizal responsiveness and an ability to extract phosphorus from soil and translocate the extracted phosphorus from the soil and into the plant when grown in soil containing mycorrhiza and have introgressed into its genome, genetic material from at least one, preferably more than one, and most preferably all, of the hereinbefore described quantitative trait loci.

The present invention also contemplates a method for introgressing into an inbred maize plant genetic material that confers upon the plant the traits of mycorrhizal responsiveness and/or the ability to extract phosphorus from soil and translocate the extracted phosphorus from the soil into the plant.

In one embodiment, the present invention relates to a method for introgressing into an inbred maize plant, genetic material that confers upon the plant the trait of mycorrhizal responsiveness when grown in soil containing mycorrhiza. When this inbred maize plant is crossed with a second inbred maize plant, it produces a hybrid maize plant which exhibits mycorrhizal responsiveness when grown in soil containing mycorrhiza. The method involves selecting a first donor maize parental line or progeny thereof which has a genome which exhibits the trait of mycorrhizal responsiveness when grown in soil containing mycorrhiza. This first donor maize parental line contains genes located in at least one of the following quantitative trait loci: chromosome 2, interval 160 to 178, chromosome 5, interval 0 to 8, chromosome 7, interval 46 to 74, and chromosome 9, interval 50 to 72.

After selection of the first donor maize parental line, a second donor maize parental line having desirable phenotypic traits is selected. Examples of the types of desirable phenotypic traits that can be exhibited by the second donor maize parental line include high yield, disease resistance, insect resistance, etc.

Once the first and second donor maize parental lines are selected, the two donor maize parental lines are crossed to produce a segregating plant population. The segregating plant population is screened for plants containing at least one of the hereinbefore described quantitative trait loci associated with the trait of mycorrhizal responsiveness. The segregating plant population can be screened using any technique known in the art, such as molecular marker screening or restriction fragment length polymorphism. Preferably, one or more of the molecular markers listed above in Table 1 above can be used to screen for plants containing the one or more of the relevant quantitative trait loci.

The plants containing at least one of the hereinbefore described quantitative trait loci are selected for further crossings and screening. These plants are crossed using techniques which are well known in the art. Additionally, these plants are screened using techniques well known in the art, such as molecular marker screening or restriction fragment length polymorphism. These plants are crossed and screened until a maize line is obtained which is homozygous for the quantitative trait loci from the first donor maize parental line. These plants are then selected. The end result of the screening and selection process is a homozygous inbred line which contains the allele for the trait of mycorrhizal responsiveness at one or more of the hereinbefore described quantitative trait loci. These selected plants can then be used as donor parents to produce hybrid plants having the trait of mycorrhizal responsiveness when grown in soil containing mycorrhiza. An example of an inbred maize line that is homozygous for all of the hereinbefore described quantitative trait loci and confers the trait of mycorrhizal responsiveness is B73.

The present invention also contemplates hybrid maize plants having a genome that contains genes which confer to the plant the trait of myccorhizal responsiveness when grown in soil containing mycorrhiza and a method for producing the hybrid maize plants. The hybrid maize plants are produced by crossing a first inbred donor maize plant, such as the one produced by the method described hereinbefore which contains within its genome at least one of the hereinbefore described quantitative trait loci, with a second inbred donor maize plant having desirable phenotypic traits, to produce a segregating population. The segregating plant population is then screened for plants containing one or more of the hereinbefore described quantitative trait loci that are associated with conferring to the plant the trait of myccorhizal responsiveness when grown in soil containing myccorhiza.

In a second embodiment, the present invention relates to a method for introgressing into an inbred maize plant genetic material that confers upon the plant an ability to extract phosphorus from soil and translocate the extracted phosphorus from the soil and into the plant. When this inbred maize plant is crossed with a second inbred maize plant, it produces a hybrid maize plant which has the ability to extract phosphorus from the soil and translocate the extracted phosphorus from the soil and into the plant. The method involves selecting a first donor maize parental line or progeny thereof which has a genome which exhibits an ability to extract phosphorus from the soil and then translocate this extracted phosphorus from the soil and into the plant. This first donor maize parental line contains the genes located in one or more of the following quantitative trait loci: chromosome 1, interval 186 to 212, chromosome 7, interval 46 to 74, chromosome 8, interval 88 to 106; and chromosome 9, interval 56 to 62. After selection of the first donor maize parental line, a second donor maize parental line having desirable phenotypic traits is selected.

Once the first and second donor maize parental lines are selected, the two donor maize parental lines are crossed to produce a segregating plant population. The segregating plant population is then screened for plants containing one or more of the hereinbefore described quantitative trait loci as being associated with conferring to the plant an ability to extract phosphorus from the soil and translocate the extracted phosphorus in the plant. The segregating plant population can be screened using any technique known in the art.

The plants containing one or more of the quantitative trait loci described above are then selected for further crossings and screening. These plants are crossed using techniques which are well known in the art. Additionally, these plants are screened using techniques well known in the art. These plants are screened until a maize line is obtained which is homozygous for the quantitative trait loci from the first donor parental line. These plants are then selected. The end result of the screening and selection process is a homozygous inbred line which contains the allele for the ability to extract phosphorus from soil and translocate the extracted phosphorus from the soil and into the plant at one or more of the hereinbefore described quantitative trait loci. These selected plants can then be used as a donor parent to produce hybrid plants having genetic material that confers upon the plant an the ability to extract phosphorus from soil and translocate the extracted phosphorus from the soil and into the plant.

The present invention also contemplates improved hybrid maize plants having a genome that contains genes which confer to the plant an ability to extract phosphorus from the soil and translocate the extracted phosphorus from the soil and into the plant and a method for producing the hybrid maize plants. The hybrid maize plants are produced by crossing a first inbred donor maize plant, such as the one produced by the method described hereinbefore which contains within its genome at least one of the hereinbefore described quantitative trait loci, with a second inbred donor maize plant having desirable phenotypic traits, to produce a segregating population. The segregating plant population is then screened for plants containing one or more of the hereinbefore described quantitative trait loci that are associated with conferring to the plant an ability to extract phosphorus from the soil and translocate the extracted phosphorus from the soil into the plant. An example of an inbred maize line that is homozygous for all of the hereinbefore described quantitative trait loci and confers the trait of enabling a plant to extract phosphorus from the soil and translocate the extract phosphorus from the soil is Mo17.

In a third embodiment, the present invention relates to a method of introgressing into an inbred maize plant, genetic material which confers upon the plant the traits of mycorrhizal responsiveness and an ability to extract phosphorus from soil and translocate the extracted phosphorus from the soil into the plant when grown in soil containing mycorrhiza. When this inbred maize plant is crossed with a second inbred maize plant, it produces a hybrid maize plant which exhibits mycorrhizal responsiveness and an ability to extract phosphorus from the soil and translocate the extracted phosphorus from the soil and into the plant when grown in soil containing mycorrhiza. The method involves selecting a first donor maize parental line or progeny thereof which has a genome which exhibits mycorrhizal responsiveness when grown in soil containing mycorrhiza. This first donor maize parental line contains the genes for mycorrihiza responsiveness located on one or more of the following quantitative trait loci: chromosome 2, interval 160 to 178, chromosome 5, interval 0 to 8, chromosome 7, interval 46 to 74, and chromosome 9, interval 50 to 72.

After selection of the first donor inbred maize parental line, a second donor inbred maize parental line having the ability to extract phosphorus from the soil and then translocate the extracted phosphorus from the soil and into the plant is selected. This second donor maize inbred parental line contains the genes located on one or more of the following quantitative trait loci: chromosome 1, interval 186 to 212, chromosome 7, interval 46 to 74, chromosome 8, interval 88 to 106, and chromosome 9, interval 56 to 62.

Once the first and second donor maize parental lines are selected, the two donor maize parental lines are crossed to produce a segregating plant population. The segregating plant population is then screened for plants containing one or more of the hereinbefore described quantitative trait loci as being associated mycorrhizal responsiveness and the ability to extract phosphorus from the soil and translocate the extracted phosphorus from the soil and into the plant when grown in soil containing mycorrhiza. The segregating plant population can be screened using any technique known in the art.

The plants containing one or more of the quantitative trait loci described above are then selected for further crossing and screening. These plants are crossed using techniques which are well known in the art. Additionally, these plants are screened using techniques well known in the art. These plants are crossed and screened until a maize line is obtained which is homozygous for the quantitative trait loci from the donor parental lines which confer the traits of mycorrhizal responsiveness and the ability to extract phosphorus from the soil and translocate the extracted phosphorus from the soil and into the plant. These plants are then selected. The end result of the screening and selection process is a homozygous inbred line which contains the alleles for the traits of mycorrhizal responsiveness and the ability to extract phosphorus from soil and translocate the extracted phosphorus from the soil and into the plant at one or more of the hereinbefore described quantitative trait loci. These selected plants can then be used as donor parents to produce improved hybrid plants having enhanced genetic material that confers upon the plant the traits of mycorrhizal responsiveness and the ability to extract phosphorus from soil and translocate the extracted phosphorus from the soil and into the plant when grown in soil containing mycorrhiza.

The present invention also contemplates improved hybrid maize plants and progeny thereof having a genome that contains genes which confer to the plant the traits of mycorrhizal responsiveness and an ability to extract phosphorus from the soil and translocate the extracted phosphorus from the soil when grown in soil containing mycorrhiza and a method for producing such improved hybrid maize plants. The hybrid maize plants are produced by crossing a first inbred maize parental line, such as the inbred line produced by the method described hereinbefore which contains within its genome at least one of the hereinbefore described quantitative trait loci which are associated with the ability to extract phosphorus from the soil and translocate the extracted phosphorus from the soil and into the plant, with a second inbred maize parental line, such as an inbred line produced by the method described hereinbefore which contains within its genome at least one of the hereinbefore described quantitative trait loci which are associated with mycorrhizal responsiveness, to produce a segregating population.

Additionally, the improved hybrid maize plant can be produced by crossing a first inbred maize parental line, such as an inbred line produced by the method described hereinbefore, which contains within its genome at least one of the hereinbefore described quantitative trait loci which are associated with the trait of mycorrhizal responsiveness and the ability to extract phosphorus form the soil and translocate the extracted phosphorus from the soil and into the plant with a second inbred donor maize parental line exhibiting desirable phenotypic traits. The segregating plant population is then screened for plants containing one or more of the hereinbefore described quantitative trait loci that are associated with conferring to the plant the traits of mycorrhizal responsiveness and the ability to extract phosphorus from the soil and translocate the extracted phosphorus from the soil into the plant when grown in soil containing mycorrhiza.

By way of example, and not of limitation, examples of the invention will now be given.

### EXAMPLE 1

### Materials and Methods

Genotypes were evaluated using a glasshouse analysis procedure to assess responsiveness to mycorrhizae, and growth under high and low phosphorus conditions. “High phosphorus conditions” as used the examples refers to soil contain about 167 mg kg⁻¹. “Low phosphorus conditions” as used the examples refers to soil containing about 17 mg kg⁻¹. Each inbred genotype was planted in four treatments: 1) low phosphorus conditions with no mycorrhizae, 2) low phosphorus conditions plus mycorrhizal inoculum, 3) high phosphorus conditions with no mycorrhizae, and 4) high phosphorus conditions plus mycorrhizal inoculum. The low phosphorus soil was obtained from the University of Wisconsin Marshfield Experiment Station. This soil is a Marshfield silt loam having a pH 6.5 and is non-calcareous. Soil phosphorus levels, and nutrient levels in general, were depleted in this soil by continuous planting of corn without fertilization for greater than 15 years. Three replicates of plants were grown in 500 ml containers per treatment, for a total of twelve plants of each genotype analyzed. The mycorrhizal inoculum was a mixture of five species of mycorrhizae obtained from the International Culture Collection of Arbuscular and VA Mycorrhizal Fungi (INVAM), West Virginia University. The species are Glomus etunicatum UT316, G. claroideum IN113, G. clarum and Acaulospora mellea BR147, G intraradices ON103, and Gigaspora rosea FL105.

Plants were grown in a temperature controlled glasshouse maintained at about 27° C. and fertilized biweekly with Peters 25-0-25 fertilizer. Six weeks after emergence, plants were harvested and plant dry weight and root volume measured for all treatments. In addition, root samples were cleared and stained to determine percent of root colonized, and to ascertain that plants grown in sterile soil did not form mycorrhizae. Mycorrhizal responsiveness was calculated from the plants grown at low phosphorus conditions as follows: [(shoot weight mycorrhizal—shoot weight non-mycorrhizal)×100]/shoot weight non-mycorrhizal (See Hetrick et al., Can. J. Bot., 70:2032-2040 (1992), herein incorporated by reference). Forty-two public maize inbred genotypes were analyzed in the study representing a range of genetic variation for corn grown in the Midwest; the values for twelve inbreds representing the full range of response are shown below in Table 2.

Based on the inbred screening, B73 was chosen as a genotype which grew the poorest in soil under low phosphorus conditions and had a genetic capacity to respond to mycorrhizal inoculation. Mo17 was chosen as a genotype which grew the best in the soil under low phosphorus conditions, and had a genetic capacity to respond to mycorrhiza. The publicly available B73×Mo17 recombinant inbred population provided by C. Stuber and L. Senior, USDA and North Carolina State University was used to map genes controlling an enhanced ability to extract phosphorus from the soil and translocate the extracted phosphorus from the soil and into the plant and mycorrhiza responsiveness in this cross.

### EXAMPLE 2

### Genetic Mapping of Loci involved in Mycorrhizal Responsiveness and Extraction and Translocation of Phosphorus from the Soil in a RI population of B73×Mo17

One hundred ninety-six recombinant inbred families were grown in the glasshouse as described above, except that the high phosphorus, mycorrhizal treatment was omitted. Shoot dry weight at low phosphorus conditions without mycorrhiza (referred to as “LOW”) and percent response to mycorrhiza (referred to as “RESPONSE”) (calculated as [(shoot weight mycorrhizal—shoot weight non-mycorrhizal)×100]/shoot weight non-mycorrhizal) were the variables used in the genetic mapping analysis.

One hundred ninety-six molecular markers, comprised of eight isozyme, 125 RFLP, and 33 microsattelite loci, were used in the genetic analysis. This data was supplied by C. Stuber and L. Senior, and are publicly available on the Maize Genome Database maintained by the National Agriculture Library in Washington D.C.

Recombinant inbred lines and replicates were considered random effects; treatments and marker genotypes were fixed effects. Mycorrhizal responsiveness was calculated as described above. Linkages between molecular markers and quantitative trait loci were determined in a two-step process using PlabQTL (Utz, et al., J. Quant. Trait Loci: 2:1 (1996), herein incorporated by reference). First, an analysis was done using simple interval mapping. Next, the most significant marker in each peak greater than LOD 1.0 was used as a cofactor for composite interval mapping. All chromosome regions with LOD>1.5 from the composite analysis were considered significant and included in the final multiple regression model. The additive effect of a marker was calculated as [(mean of the homozygous Mo17 class—mean of the homozygous B73 class)/2].

Four chromosome regions were determined to contain QTL for shoot weight of plants grown at low phosphorus in the absence of mycorrhiza (LOW) (See Table 3a). A region of chromosome 8 near marker phi060 had the largest additive effect. This region of chromosome 8 has also been found to have an effect on plant maturity in several populations (See Phillips et al., Proc. 47th Annual Corn & Sorghum Indust. Res. Conf., 135-150(1997) and Koester et al., Crop Sci., 33:1209-1216 (1993)) and was found to be significant for days to pollen shed in this population. Significant QTL were also detected on chromosomes 1, 7, and 9. The allele from Mo17 had a positive effect on LOW for all four QTL detected, consistent with it being the best growing parent under low P conditions.

Four QTL were detected for the trait RESPONSE (See Table 3b). The RESPONSE and LOW QTL on chromosome 7 and 9 had overlapping confidence intervals, consistent with the inventors hypothesis that a gene(s) in a single chromosome region controls both traits. Favorable alleles for RESPONSE came from B73 at the chromosome 5, 7, and 9 QTL and from Mo17 at the chromosome 2 QTL. Digenic epistasis was not detected among QTL for LOW or RESPONSE (p<0.05).

Table 2. Dry weights of 28 maize inbred lines and mycorrhizal responsiveness. Data is sorted by growth at low phosphorus in the absence of mycorrhiza (M−P−). Means at bottom of columns are given across 42 inbreds included in this study (all data not shown, but extremes are presented). The experiment was a factorial for presence (M+) or absence (M−) of mycorrhizae, and high phosphorus (P+, 167 ppm) and low phosphorus (P−, 17 ppm).

| | | M + P− | Mycorrhizal |
|---|---|---|---|
| Shoot weight† | Response to | percent of | benefit at |

| | | | | | | | |
|---|---|---|---|---|---|---|---|
| INBRED | M − P− | M + P− | M − P+ | M + P+ | mycorrhiza‡ | control§ | high P¶ |

| | |
|---|---|
| g | % |

| | | | | | | | |
|---|---|---|---|---|---|---|---|
| Mo17 | 3.15 | 5.23 | 11.96 | 7.96 | 66 | 66 | 67 |
| We10 | 2.24 | 4.16 | 10.64 | 11.33 | 86 | 37 | 106 |
| B79 | 2.18 | 5.53 | 11.71 | 11.41 | 154 | 48 | 97 |
| Oh40B | 2.16 | 4.76 | 11.65 | 8.97 | 120 | 53 | 97 |
| B37 | 1.97 | 5.17 | 11.65 | 8.96 | 162 | 58 | 77 |
| Pa32 | 1.96 | 5.28 | 10.36 | 9.60 | 169 | 55 | 93 |
| A619 | 1.81 | 4.30 | 9.08 | 9.94 | 138 | 43 | 109 |
| H99 | 1.73 | 4.52 | 10.75 | 11.13 | 161 | 41 | 104 |
| Wf9 | 1.69 | 3.97 | 10.10 | 9.74 | 135 | 41 | 96 |
| N28E | 1.65 | 3.93 | 8.43 | 7.15 | 138 | 55 | 85 |
| WH | 1.57 | 3.51 | 15.01 | 10.13 | 124 | 35 | 67 |
| NY821 | 1.51 | 5.42 | 8.78 | 10.50 | 259 | 52 | 120 |
| B84 | 1.49 | 3.93 | 11.33 | 9.76 | 164 | 40 | 86 |
| W59M | 1.45 | 6.04 | 13.03 | 12.62 | 317 | 48 | 93 |
| A632 | 1.36 | 5.09 | 16.08 | 12.97 | 274 | 39 | 77 |
| W37A | 1.31 | 6.19 | 13.06 | 11.57 | 373 | 54 | 89 |
| IA4189 | 1.15 | 4.67 | 8.60 | 8.91 | 306 | 52 | 104 |
| A188 | 0.97 | 4.78 | 12.49 | 13.75 | 393 | 35 | 110 |
| N28 | 0.93 | 3.41 | 7.64 | 7.62 | 267 | 45 | 100 |
| B14 | 0.85 | 4.25 | 12.25 | 8.26 | 400 | 51 | 67 |
| MS1334 | 0.83 | 3.65 | 9.63 | 5.91 | 340 | 62 | 61 |
| Oh43 | 0.78 | 5.79 | 11.11 | 9.61 | 642 | 60 | 86 |
| W64A | 0.73 | 3.99 | 10.20 | 9.52 | 447 | 42 | 81 |
| Pa36 | 0.70 | 1.95 | 10.73 | 10.03 | 179 | 19 | 93 |
| CMD5 | 0.65 | 3.82 | 13.13 | 8.31 | 488 | 46 | 63 |
| HiIIB | 0.59 | 4.33 | 11.81 | 7.15 | 634 | 61 | 61 |
| B73 | 0.58 | 4.37 | 11.35 | 9.23 | 653 | 47 | 81 |
| HiIIA | 0.56 | 3.26 | 10.85 | 8.95 | 482 | 36 | 82 |
| MEAN | 1.4 | 4.5 | 11.3 | 9.9 | 282 | 47 | 88 |
| LSD _{(0.05)} | 1.0 | 1.4 | 2.9 | 2.6 | | | |

†M − P−: low P, no mycorrhiza; M + P−: low P, mycorrhiza; M − P+: high P, no mycorrhiza; M + P+: high P, mycorrhiza
‡[(M + P− - M − P−)/M − P−] x 100
§(M + P−/M + P+) x 100
¶(M + P+/M − P+) x 100

TABLE 3
Summary of QTL controlling maize growth at low P in the absence of
mycorrhiza (LOW) and maize growth response to mycorrhizal inocuation
(RESPONSE). QTL analysis based on shoot dry weight from a greenhouse
analysis of 198 B73xMo17 recombinant inbred lines harvested
approximately eight weeks after planting.
Table 3a - LOW

| | | | Position | Additive |
|---|---|---|---|---|
| QTL | Closest | | (confidence | effect‡ |
| Designation | marker | Chromosome | interval)† | g |
| LOW1 | UMC107a | 1 | 200 (186 to 212) | 0.070 |
| LOW2 | UMC110a | 7 | 64 (46 to 74) | 0.071 |
| LOW3 | PHI060 | 8 | 96 (88 to 106) | 0.075 |
| LOW4 | PHI130 | 9 | 60 (56 to 62) | 0.071 |

†LOD_{1.0} confidence interval. Values for position and confidence interval relate to FIG. 1.
‡Calculated based on marker allele means as (Mo17 homozygote mean - B73 homozygote mean)/2. Therefore, positive values indicate that Mo17 contains a favorable allele.

TABLE 3b
RESPONSE

| | | | Position | Additive |
|---|---|---|---|---|
| QTL | Closest | | (confidence | effect‡ |
| Designation | marker | Chromosome | interval)† | % |
| RESPONSE1 | UMC49a | 2 | 174 (160 to 178) | 33.6 |
| RESPONSE2 | NPI409 | 5 | 0 (end to 8) | −33.43 |
| RESPONSE3 | UMC110a | 7 | 66 (46 to 74) | −32.68 |
| RESPONSE4 | PHI061 | 9 | 62 (50 to 72) | −62.62 |

†LOD_{1.0} confidence interval. Values for position and confidence interval relate to FIG. 1.
‡Calculated based on marker allele means as (Mo17 homozygote mean - B73 homozygote mean)/2. Therefore, positive values indicate that Mo17 contains a favorable allele.

What is claimed is:

1. A maize plant having a genome containing quantitative trait loci which confer to the plant an ability to extract phosphorus from soil and translocate the extracted phosphorus into the plant wherein the quantitative trait loci are located on chromosome 1, interval 186 to 212, chromosome 7, interval 46 to 74, chromosome 8, interval 88 to 106, and chromosome 9, interval 56 to 62.

2. A maize plant having a genome containing quantitative trait loci which confer to the plant mycorrhizal responsiveness when the plant is grown in soil containing mycorrhiza, wherein the quantitative trait loci are located on chromosome 2, interval 160 to 178, chromosome 5, interval 0 to 8, chromosome 7, interval 46 to 74, and chromosome 9, interval 50 to 72.

3. A maize plant having a genome containing quantitative trait loci which confer to the plant mycorrhizal responsiveness and an ability to extract phosphorus from soil and translocate the extracted phosphorus into the plant when the plant is grown in soil containing mycorrhiza, wherein the quantitative trait loci are located on chromosome 2, interval 160 to 178, chromosome 5, interval 0 to 8, chromosome 7, interval 46 to 74, and chromosome 9, interval 50 to 72, for mycorrhizal responsiveness, and on chromosome 1, interval 186 to 212, chromosome 7, interval 46 to 74, chromosome 8, interval 88 to 106, and chromosome 9, interval 56 to 62 for the ability to extract phosphorus from soil and translocate phosphorus into the plant.

4. A maize plant having a genome which confers to the plant an ability to extract phosphorus from soil and translocate the extracted phosphorus into the plant, wherein the genome contains quantitative trait loci from a first donor maize line parent and genes associated with the ability to extract phosphorus from the soil and translocate the extracted phosphorus into the plant from a second donor maize line parent, wherein the quantitative trait loci in the second donor maize line parent are located on chromosome 1, in the genetic map interval 186 to 212, chromosome 7, interval 46 to 74, chromosome 8, interval 88 to 106, and chromosome 9, interval 56 to 62.

5. The maize plant of claim 4 wherein the second donor maize line parent is Mo17 or progeny thereof.

6. A maize plant having a genome which confers to the plant mycorrhizal responsiveness when the plant is grown in soil containing mycorrhiza, wherein the genome contains quantitative trait loci from a first donor maize line parent and genes associated with mycorrhizal responsiveness from a second donor maize line parent, wherein the quantitative trait loci in the second donor maize parent line are located on chromosome 2, interval 160 to 178, chromosome 5, interval 0 to 8, chromosome 7, interval 46 to 74, and chromosome 9, interval 50 to 72.

7. The maize plant of claim 6 wherein the second donor maize line parent is B73 or progeny thereof.

8. A maize plant having a genome which confers to the plant mycorrhizal responsiveness and an ability to extract phosphorus from soil and translocate the extracted phosphorus into the plant when the plant is grown in soil containing mycorrhiza, wherein the genome contains quantitative trait loci associated with mycorrhizal responsiveness from a first donor maize line parent B73, or progeny thereof, and quantitative trait loci associated with an ability to extract phosphorus from soil and translocate the extracted phosphorus into the plant from a second donor maize line parent Mo17, or progeny thereof.

9. The maize plant of claim 8 wherein the quantitative trait loci associated with mycorrhizal responsiveness are located on chromosome 2, interval 160 to 178, chromosome 5, interval 0 to 8, chromosome 7, interval 46 to 74, and chromosome 9, interval 50 to 72.

10. The maize plant of claim 8 wherein the quantitative trait loci associated with the ability of the plant to extract phosphorus from the soil and translocate the extracted phosphorus into the plant, are located on chromosome 1, interval 186 to 212, chromosome 7, interval 46 to 74, chromosome 8, interval 88 to 106 and chromosome 9, interval 56 to 62.

11. A method for producing an inbred maize plant, which when crossed with a second inbred maize plant, produces a hybrid maize plant which has an ability to extract phosphorus from soil and translocate the extracted phosphorus into the plant, the method comprising the steps of:
a. selecting a first maize parental line which has a genome which exhibits an ability to extract phosphorus from soil and translocate the extracted phosphorus into the plant and contains quantitative trait loci located on chromosome 1, interval 186 to 212, chromosome 7, interval 46 to 74, chromosome 8, interval 88 to 106, and chromosome 9, interval 56 to 62;
b. selecting a second maize parental line having desirable phenotypic traits;
c. crossing the first maize parental line with the second donor maize parental line to produce a segregating plant population;
d. screening the plant population for one or more quantitative trait loci associated with the ability to extract phosphorus from the soil and translocate the extracted phosphorus into the plant; and
e. selecting plants from the population having the identified quantitative trait loci for further screening until a maize line is obtained which is homozygous for the ability to extract phosphorus from the soil and translocate the extracted phosphorus into the plant at sufficient quantitative trait loci to give a hybrid maize plant having an ability to extract phosphorus from the soil and translocate the extracted phosphorus into the plant.

12. The method of claim 11 wherein the first maize parental line is Mo17.

13. An inbred maize plant produced by the method of claim 11.

14. A method for producing a hybrid maize plant having a genome containing quantitative trait loci which confer to the plant an ability to extract phosphorus from soil and translocate the extracted phosphorus into the plant, the method comprising the steps of:
a. crossing the inbred maize plant of claim 9 with a maize inbred line having desirable phenotypic traits to produce a segregating plant population;
b. screening the plant population for one or more quantitative trait loci associated with the ability to extract phosphorus from the soil and translocate the extracted phosphorus into the plant; and
c. selecting hybrid plants from the population exhibiting the ability to extract phosphorus from the soil and translocate the extracted phosphorus into the plant.

15. A hybrid maize plant produced by the method of claim 14.

16. A method for producing an inbred maize plant, which when crossed with a second inbred maize plant, produces a hybrid maize plant which exhibits mycorrhizal responsiveness when grown in soil containing mycorrhiza, the method comprising the steps of:
a. selecting a first maize parental line which has a genome which exhibits mycorrhizal responsiveness and contains quantitative trait loci located chromosome 2, interval 160 to 178, chromosome 5, interval 0 to 8, chromosome 7, interval 46 to 74, and chromosome 9 interval 50 to 72;
b. selecting a second donor maize parental line having desirable phenotypic traits;
c. crossing the first maize parental line with the second donor maize parental line to produce a segregating plant population;
d. screening the plant population for one or more quantitative trait loci associated mycorrhizal responsiveness; and
e. selecting plants from the population having the identified quantitative trait loci for further screening until a maize line is obtained which is homozygous for mycorrhizal responsiveness at sufficient quantitative trait loci to give a hybrid maize plant having mycorrhizal responsiveness.

17. The method of claim 16 wherein the first maize parental line is B73.

18. An inbred maize plant produced by the method of claim 16.

19. A method for producing a hybrid maize plant having a genome containing quantitative trait loci which confer to the plant mycorrhizal responsiveness when the plant is grown in soil containing mycorrhiza, the method comprising the steps of:
a. crossing the inbred maize plant of claim 18 with a maize inbred line having desirable phenotypic traits to produce a segregating plant population;
b. screening the plant population for one or more quantitative trait loci associated with mycorrhizal responsiveness; and
c. selecting hybrid plants from the population exhibiting mycorrhizal responsiveness.

20. A hybrid maize plant produced by the method of claim 19.

21. A method for producing an inbred maize plant, which when crossed with a second inbred maize plant, produces a hybrid maize plant which exhibits the traits of mycorrhizal responsiveness and an ability to extract phosphorus from the soil and translocate the extracted phosphorus into the plant when the plant is grown in soil containing mycorrhiza, the method comprising the steps of:
a. selecting a first maize parental line having a genome which exhibits an ability to extract phosphorus from soil and translocate the extracted phosphorus into the plant and contains quantitative trait loci located on chromosome 1, interval 186 to 212, chromosome 7, interval 46 to 74, chromosome 8, interval 88 to 106, and chromosome 9, interval 56 to 62;
b. selecting a second maize parental line having a genome which exhibits mycorrhizal responsiveness when grown in soil containing mycorrhiza and contains quantitative trait loci located on chromosome 2, interval 160 to 178, chromosome 5, interval 0 to 8, chromosome 7, interval 46 to 74, and chromosome 9, interval 50 to 72;
c. crossing the first maize parental line with the second maize parental line to produce a segregating plant population;
d. screening the plant population for one or more quantitative trait loci associated with mycorrhizal responsiveness and the ability to extract phosphorus from the soil and translocate the extracted phosphorus into the plant; and
e. selecting plants from the population having the identified quantitative trait loci for further screening until a maize line is obtained which is homozygous for the traits of mycorrhizal responsiveness and the ability to extract phosphorus from the soil and translocate the extracted phosphorus into the plant at sufficient quantitative trait loci to give a hybrid maize plant having the traits of mycorrhizal responsiveness and the ability to extract phosphorus from the soil and translocate the extracted phosphorus into the plant, when grown in soil containing mycorrhiza.

22. The method of claim 21 wherein the first maize parental line is Mo17.

23. The method of claim 21 wherein the second maize parental line is B73.

24. An inbred maize plant produced by the process of claim 21.

25. A method for producing a hybrid maize plant having a genome containing quantitative trait loci which confer to the plant the traits of mycorrhizal responsiveness and an ability to extract phosphorus from soil and translocate the extracted phosphorus from the soil and into the plant when grown in soil containing mycorrhiza, the method comprising the steps of:
a. crossing the inbred maize plant of claim 24 with a maize inbred line having desirable phenotypic traits to produce a segregating plant population;
b. screening the plant population for one or more quantitative trait loci associated with the traits of mycorrhizal responsiveness and the ability to extract phosphorus from the soil and translocate the extracted phosphorus from the soil and into the plants; and
c. selecting hybrid plants from the population having the traits mycorrhizal responsiveness and the ability to extract phosphorus from the soil and translocate the extracted phosphorus from the soil and into the plant.

26. A hybrid maize plant produced by the process of claim 25.

27. A method for producing a hybrid maize plant having a genome containing quantitative trait loci which confer to the plant the traits of mycorrhizal responsiveness and an ability to extract phosphorus from soil and translocate the extracted phosphorus and into the plant when grown in soil containing mycorrhiza, the method comprising the steps of:
a. crossing the inbred maize plant of claim 13 with the inbred maize plant of claim 18 to produced a segregating plant population;
b. screening the plant population for one or more quantitative trait loci associated with the traits of mycorrhizal responsiveness and the ability to extract phosphorus from the soil and translocate the extracted phosphorus from the soil and into the plant; and
c. selecting plants from the population having the traits of mycorrhizal responsiveness and an ability to extract phosphorus from the soil and translocate the extracted phosphorus from the soil and into the plant.

28. A hybrid maize plant produced by the process of claim 27.
